(19) United States
(12) Patent Application Publication
(10) Pub. No.: US 2026/0241186 A1
(43) Pub. Date: Aug. 20, 2026

(54) RADIO FREQUENCY BEAUTY INSTRUMENT

(71) Applicant: PEGATRON CORPORATION, TAIPEI CITY (TW)

(72) Inventors: Shih-Po Liu, Taipei City (TW); Chun-Liang Yeh, Taipei City (TW); Tsu-Hsuan Huang, Taipei City (TW); An-Chun Chiang, Taipei City (TW)

(73) Assignee: PEGATRON CORPORATION, TAIPEI CITY (TW)

(21) Appl. No.: 19/382,190

(22) Filed: Nov. 6, 2025

(30) Foreign Application Priority Data
Feb. 14, 2025 (TW) 114105538

(51) Int. Cl. A61N 1/40; A61N 1/06; A61N 1/32

(52) U.S. Cl. CPC A61N 1/40; A61N 1/06; A61N 1/328

(57) ABSTRACT

A radio frequency beauty instrument is suitable for using on skin of a user. The radio frequency beauty instrument includes a housing, a main circuit board, a gear set, a drive unit, and a radio frequency module. The main circuit board is disposed in the housing. The gear set is disposed in the housing. The drive unit is disposed in the housing and electrically connected to the main circuit board. The drive unit is configured to drive the gear set. The radio frequency module includes a radio frequency circuit disposed on the main circuit board and multiple electrode units exposed outside the housing and electrically connected to the radio frequency circuit. The electrode units are connected to the gear set to be driven by the gear set to rotate. The radio frequency module provides a radio frequency current to the skin through the radio frequency circuit and the electrode units.

### CROSS-REFERENCE TO RELATED APPLICATION

This application claims the priority benefit of Taiwan application serial no. 114105538, filed on February 14, 2025. The entirety of the above-mentioned patent application is hereby incorporated by reference herein and made a part of this specification.

### BACKGROUND

### Technical Field

The disclosure relates to a radio frequency beauty instrument.

### Related Art

With technological advancement, radio frequency beauty instruments have been launched in the market, utilizing radio frequency technology through generated high-frequency current to make skin tissue generate heat, stimulating the proliferation and contraction of collagen, thereby achieving the effect of tightening skin and reducing wrinkles. How to enhance the convenience of radio frequency beauty instruments is a research direction in this field.

### SUMMARY

The disclosure provides a radio frequency beauty instrument, which may enhance the convenience of radio frequency beauty instruments.

A radio frequency beauty instrument of the disclosure is suitable for using on the skin of a user, the radio frequency beauty instrument includes a housing, a main circuit board, a gear set, a drive unit, and a radio frequency module. The main circuit board is disposed in the housing. The gear set is disposed in the housing. The drive unit is disposed in the housing and electrically connected to the main circuit board, and the drive unit is configured to drive the gear set. The radio frequency module includes a radio frequency circuit disposed on the main circuit board and a plurality of electrode units exposed outside the housing and electrically connected to the radio frequency circuit. The electrode units are connected to the gear set to be driven by the gear set to rotate, and the radio frequency module provides a radio frequency current to the skin through the radio frequency circuit and these electrode units.

In an embodiment of the disclosure, the electrode units respectively include a plurality of electrode disk bodies and a plurality of electrode protrusions located on the electrode disk bodies, and when the electrode units are driven by the gear set to rotate, a distance between the electrode protrusions changes.

In an embodiment of the disclosure, two of the electrode disk bodies have different diameters.

In an embodiment of the disclosure, two of the electrode disk bodies have the same diameter.

In an embodiment of the disclosure, the gear set includes a first gear and a plurality of second gears meshing with the first gear, the first gear is a driving gear, these second gears are driven gears, the drive unit drives the first gear, and the second gears are respectively driven to rotate the electrode units.

In an embodiment of the disclosure, the drive unit and the first gear are coaxial, and each of the second gears is coaxial with the corresponding electrode unit.

In an embodiment of the disclosure, the second gears surround the first gear, and the rotation direction of the first gear is opposite to the rotation direction of the second gears.

In an embodiment of the disclosure, the radio frequency beauty instrument further includes a sub circuit board electrically connected to the main circuit board, and disposed between the gear set and these electrode units.

In an embodiment of the disclosure, each of the electrode units includes at least one spring pin connector, and each of the electrode units is electrically connected to the sub circuit board by abutting the at least one spring pin connector against the sub circuit.

In an embodiment of the disclosure, the sub circuit board includes a plurality of ring pads, the ring pads respectively include at least one ring circuit, the ring circuit is a trajectory for each of the electrode units when rotating; when the electrode units are driven, the spring pin connector of each of the electrode units is rotated along the corresponding ring circuit.

Based on the above, the radio frequency module of the radio frequency beauty instrument of the disclosure provides radio frequency current to the skin through the radio frequency circuit and the electrode units to make skin tissue generate heat. Furthermore, the main circuit board is electrically connected to the drive unit, the drive unit is coupled to the gear set, and the electrode units are connected to the gear set to be driven by the gear set to rotate, so as to respond to different usage scenarios and increase convenience of use.

### BRIEF DESCRIPTION OF THE DRAWINGS

FIG. 1 is a schematic diagram showing an appearance of a radio frequency beauty instrument according to an embodiment of the disclosure.

FIG. 2A is a perspective schematic diagram of FIG. 1.

FIG. 2B is a partial exploded view of FIG. 1 with the housing omitted.

FIG. 3 to FIG. 6 are partial schematic diagrams of FIG. 2A from different viewing angles.

FIG. 7 is a schematic diagram of a sub circuit board of the radio frequency beauty instrument in FIG. 1.

FIG. 8 to FIG. 11 are schematic diagrams of a rotation process of electrode units of the radio frequency beauty instrument in FIG. 1.

### DESCRIPTION OF THE EMBODIMENTS

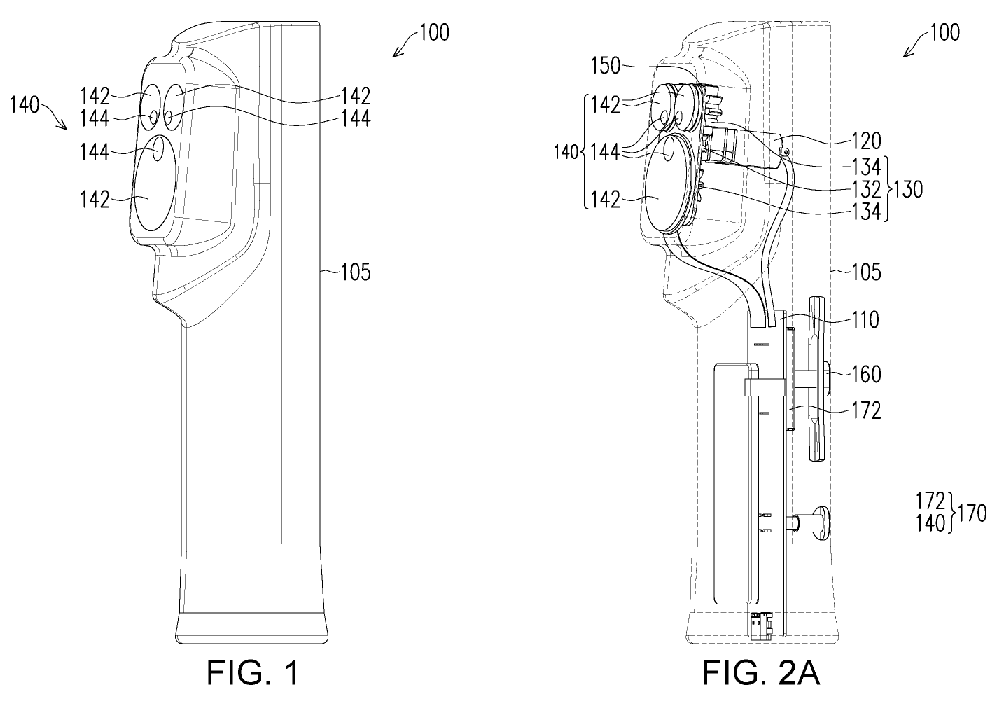

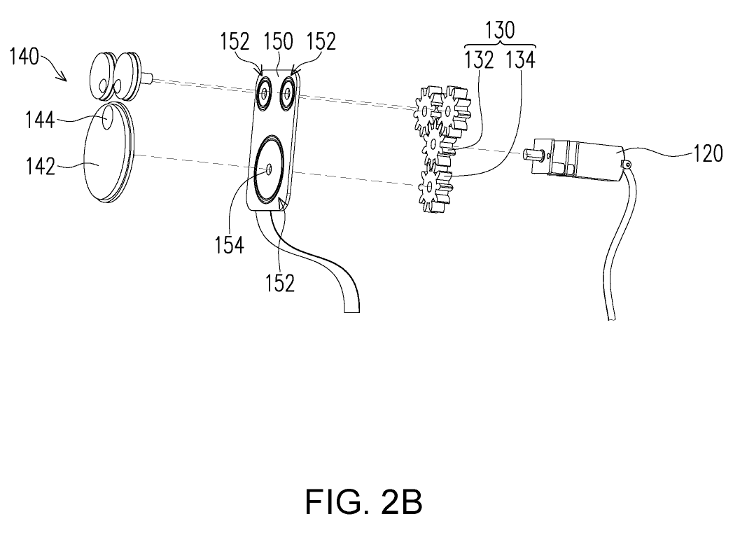

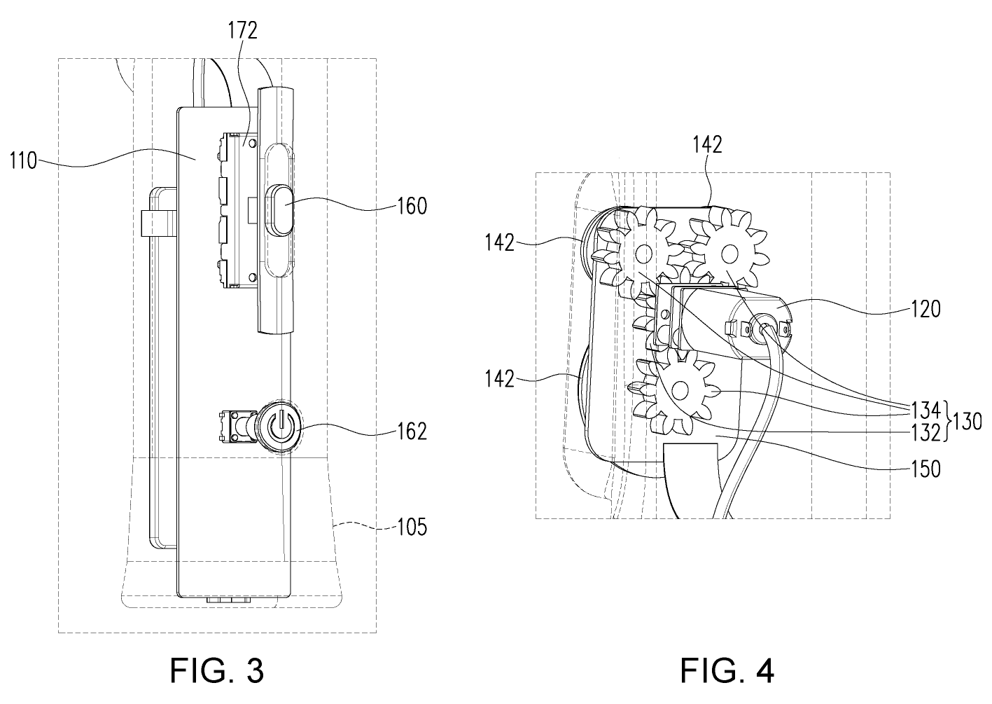

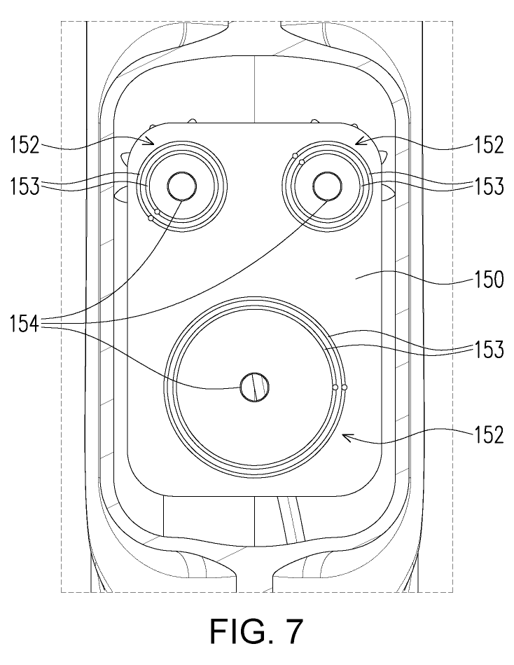

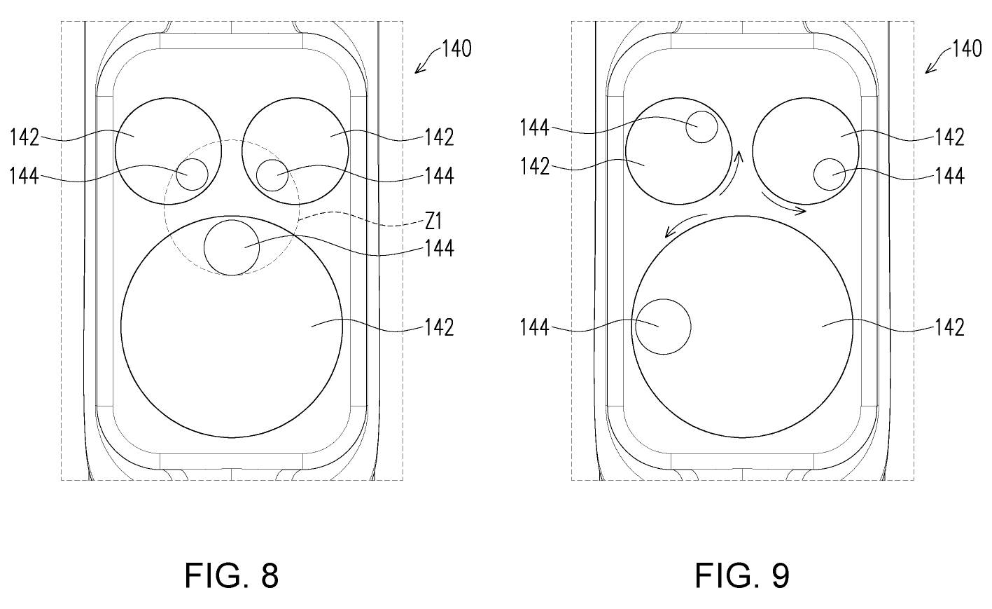

FIG. 1 is a schematic diagram showing an appearance of a radio frequency beauty instrument according to an embodiment of the disclosure. FIG. 2A is a perspective schematic diagram of FIG. 1. FIG. 2B is a partial exploded view of FIG. 1 with the housing omitted. FIG. 3 to FIG. 6 are partial schematic diagrams of FIG. 2A from different viewing angles. FIG. 7 is a schematic diagram of a sub circuit board of the radio frequency beauty instrument in FIG. 1. FIG. 8 to FIG. 11 are schematic diagrams of a rotation process of electrode units of the radio frequency beauty instrument in FIG. 1.

Referring to FIG. 1 to FIG. 11, a radio frequency beauty instrument 100 of the present embodiment is applied to the skin of a user, the radio frequency beauty instrument 100 may utilizing radio frequency technology through generated radio-frequency current (or called high frequency current), stimulating the proliferation and contraction of collagen. In addition, the radio frequency beauty instrument 100 of the present embodiment may adjust the range size of the primary treatment area by emitting radio frequency, and/or may provide massage effect, which will be described below.

As shown in FIG. 2A, the radio frequency beauty instrument 100 of the present embodiment includes a housing 105, a main circuit board 110, a drive unit 120, a gear set 130, and a radio frequency module 170. The main circuit board 110, the drive unit 120, and the gear set 130 are disposed in the housing 105.

The drive unit 120 is electrically connected to the main circuit board 110. The drive unit 120 is configured to drive the gear set 130. The drive unit 120 is, for example, a motor, but the type of the drive unit 120 is not limited thereto. In other embodiments, the drive unit 120 may also be, for example, a piezoelectric element or memory alloy.

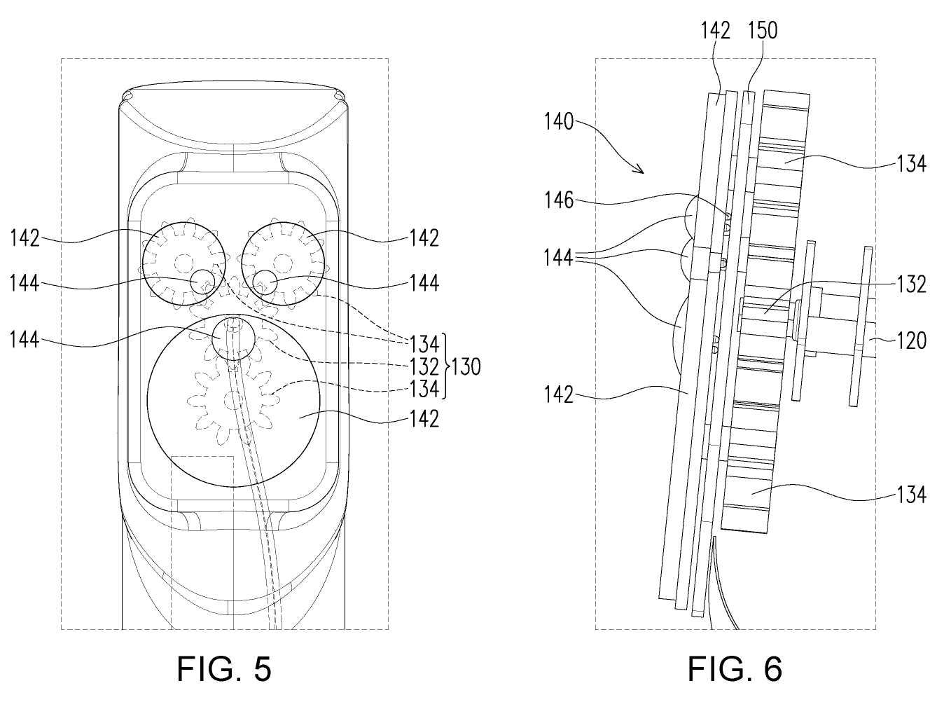

As shown in FIG. 2A and FIG. 5, the gear set 130 is coupled to the drive unit 120. Electrode units 140 are electrically connected to the main circuit board 110 and are linked to the gear set 130, and the electrode units 140 are driven by the gear set 130 to rotate.

Specifically, in the present embodiment, the gear set 130 includes a first gear 132 and a plurality of second gears 134 meshing with the first gear 132. The first gear 132 is a driving gear, and the second gears 134 are driven gears. The drive unit 120 drives the first gear 132, the first gear 132 drives the second gears 134, and the second gears 134 are respectively driven to rotate the electrode units 140.

In the present embodiment, the drive unit 120 and the first gear 132 are coaxial, and each of the second gears 134 is coaxial with the corresponding electrode unit 140. The second gears 134 surround the first gear 132, when the first gear 132 rotates, the rotation direction of the first gear 132 is opposite to the rotation direction of the second gears 134. Certainly, the configuration of the drive unit 120, the first gear 132, and the second gears 134 is not limited thereto.

In addition, in the present embodiment, the number of the second gears 134 and the number of the electrode units 140 are three as an example, but the disclosure is not limited thereto. In other embodiments, there may also be two or four or more.

The radio frequency module 170 includes a radio frequency circuit 172 disposed on the main circuit board 110 and a plurality of electrode units 140 exposed outside the housing 105 and electrically connected to the radio frequency circuit 172, and the electrode units 140 are connected to the second gears 134 of the gear set 130 to be driven by the second gears 134 of the gear set 130 to rotate. The radio frequency module 170 provides a radio frequency current to the skin through the radio frequency circuit 172 and the electrode units 140.

The electrode units 140 respectively include a plurality of electrode disk bodies 142 and a plurality of electrode protrusions 144 located on these electrode disk bodies 142. That is, each electrode unit 140 has an electrode protrusion 144 disposed on the electrode disk. The protrusion height of the electrode protrusion 144 may be designed according to requirements. In one embodiment, the electrode unit 140 may contact the skin of the user only through the electrode protrusion 144. In another embodiment, the electrode disk body 142 may also contact the skin, the electrode protrusion 144 may be the part that first contacts the skin of the user and is the main part for radio frequency current output.

As shown in FIG. 1 and FIG. 5, in the present embodiment, two of the electrode disk bodies 142 located at the upper portion have the same diameter, and the one located at the lower portion has a different diameter. Specifically, the diameter of the electrode disk body 142 located at the lower portion is larger than the diameter of the two electrode disk bodies 142 located at the upper portion. The diameter of the electrode disk body 142 located at the lower portion is, for example, twice the diameter of each electrode disk body 142 located at the upper portion, but the disclosure is not limited thereto. In other embodiments, the electrode disk bodies 142 may all have the same diameter, or may all have different diameters.

As shown in FIG. 2A and FIG. 6, in the present embodiment, the radio frequency beauty instrument 100 further includes a sub circuit board 150 electrically connected to the main circuit board 110 and disposed between the gear set 130 and the electrode units 140. The electrode units 140 are in conduction with the sub circuit board 150, and are in conduction with the main circuit board 110 through the sub circuit board 150. In the present embodiment, each of the electrode units 140 includes at least one spring pin connector 146 (FIG. 6), and each of the electrode units 140 is electrically connected to the sub circuit board 150 by abutting the at least one spring pin connector 146 against the sub circuit 150.

In addition, the sub circuit board 150 includes a plurality of through holes 154 (FIG. 7), and the electrode units 140 are respectively connected to the second gears 134 of the gear set 130 through the through holes 154.

As shown in FIG. 7, in the present embodiment, the sub circuit board 150 includes a plurality of ring pads 152, the ring pads 152 respectively include a plurality of ring circuits 153, and the ring circuits 153 are trajectories for each of the electrode units 140 when rotating; when the electrode units 140 are driven, the spring pin connector 146 of each of the electrode units 140 rotates along the corresponding ring circuit. In other embodiments, the ring pad 152 may also include only one ring circuit 153.

In the present embodiment, the ring pads 152 are, for example, three groups, the number of the ring circuits 153 of each group of ring pads 152 is, for example, two, each ring circuit 153 is a trajectory for the spring pin connector 146 corresponding to the electrode unit 140 when rotating on the ring pad 152. Therefore, by the design of the spring pin connector 146 and the ring pad 152 of the sub circuit board 150, the conduction between the electrode unit 140 and the sub circuit board 150 are still maintained when the electrode unit 140 is rotating.

In the present embodiment, the center of the electrode disk body 142 and the center of the second gear 134 are coaxial, since the spring pin connector 146 is not located at the center of the electrode disk body 142, in order to enable the electrode unit 140 to still maintain conduction with the sub circuit board 150 when rotating, the sub circuit board 150 is designed with the ring pads 152 disposed correspondingly. In other embodiments, if the position of the second gear 134 is adjusted to give way to the position corresponding to the center of the electrode disk body 142, the spring pin connector 146 may also be disposed at the center of the electrode disk body 142, in this way, the pad on the sub circuit board 150 corresponding to the spring pin connector 146 may be a non-ring pad. In one embodiment, the spring pin connector 146 and the second gear 134 may also both correspond to the center of the electrode disk body 142, and the configuration is not limited by the drawings.

In addition, in the present embodiment, as shown in FIG. 3, the radio frequency beauty instrument 100 further includes a power button 162 and an adjustment button 160 exposed from the housing, and the power button 162 and the adjustment button 160 are electrically connected to the main circuit board 110. The power button 162 is configured to turn on the operation of the radio frequency beauty instrument 100, for example, turning the instrument on/off, and turning on/off the automatic rotation mode. In the power-off condition, long pressing the power button 162 may turn on the device; in the power-on condition, long pressing the power button 162 may turn off the device. In the power-on condition, double-clicking the power button 162 may turn on the automatic rotation mode, clicking the power button 162 once may turn off the automatic rotation mode. Certainly, the function and operation method of the power button 162 are not limited thereto. The adjustment button 160 is configured to control the rotation of the drive unit 120. The user may control the adjustment button 160 by themselves to control the drive unit 120, thereby adjusting the distance between the electrode protrusions 144.

As shown in FIG. 8 to FIG. 11, when the electrode units 140 are driven to rotate by the gear set 130, the distance between the electrode protrusions 144 changes, so that the electrode protrusions 144 may adjust relative positions according to the needs of the user to vary the acting range of the radio frequency current.

Specifically, the range covered by the electrode protrusions 144 is the primary treatment area of the radio frequency current emitted by the electrode units 140. In FIG. 8, since the three electrode protrusions 144 of the three electrode units 140 are close to each other, a range Z1 (the primary treatment area of the radio frequency current emitted by these electrode units 140) covered by the three electrode protrusions 144 is minimum, which may be applied to small skin areas such as around the eyes. Since the area of the range Z1 is small, the generated heat energy may be more concentrated.

If it is desired to adjust the range covered by the three electrode protrusions 144, merely moving the adjustment button 160 is needed, the drive unit 120 drives the first gear 132 to rotate, and the second gears 134 are rotated accordingly. As shown in FIG. 9, the three electrode units 140 are correspondingly rotated, thereby changing the range covered by the three electrode protrusions 144.

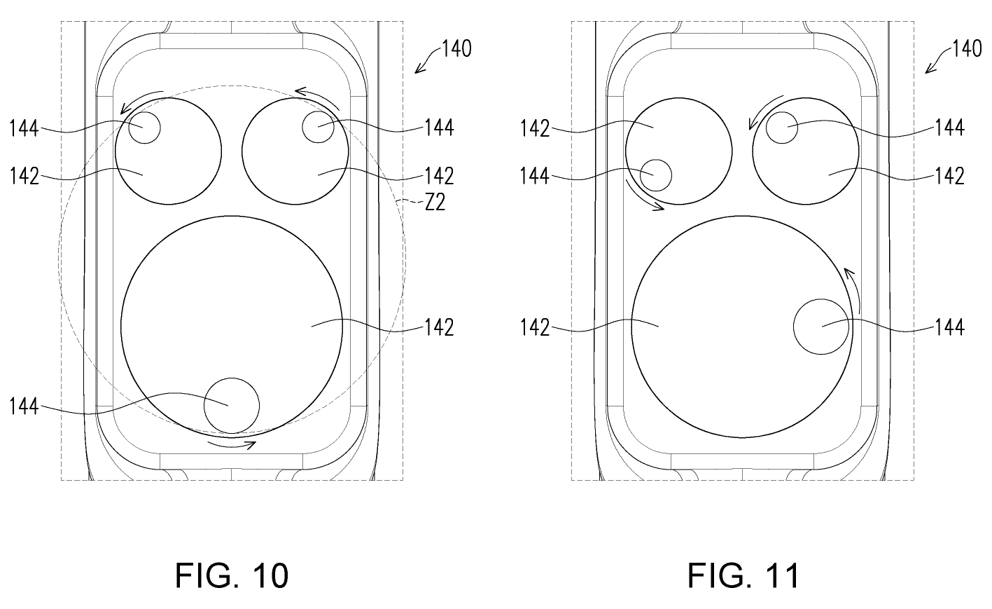

As may be seen in FIG. 10, since the three electrode protrusions 144 of the three electrode units 140 are away from each other, a range Z2 (the primary treatment area of the radio frequency current emitted by these electrode units 140) covered by the three electrode protrusions 144 is maximum, which may be applied to areas such as thighs or belly, and may complete beauty procedures on these large skin areas more quickly.

If these three electrode units 140 continue to rotate, the range covered by the three electrode protrusions 144 decreases again, and may return to the state as shown in FIG. 8. It is worth mentioning that in the automatic rotation mode, in addition to the treatment area of the radio frequency current being able to continuously change, the electrode protrusions 144 also has a massage function when these three electrode units 140 are rotating, providing the user with a soothing and relaxing effect.

Additionally, in this embodiment, each electrode unit 140 may be applied to monopolar radio frequency or multipolar radio frequency, and the type of electrode unit 140 is not limited thereto. Monopolar radio frequency has high power, acts deeper, and has higher heating temperature, being able to directly reach the dermis layer, or even the SMAS fascia layer, stimulating fibroblasts to produce more collagen regeneration; making the skin plump and tight, with significant effects and long maintenance time. Multipolar radio frequency has relatively lower and more stable energy, may reach the epidermis layer and shallow dermis layer, with more uniform distribution of acting range, being more suitable for small fine lines, puffiness, and slight skin sagging problems. Whether the electrode units 140 are applied to monopolar radio frequency or multipolar radio frequency, both may be the design of the radio frequency beauty instrument 100 in this embodiment.

In summary, the radio frequency module of the radio frequency beauty instrument of the disclosure provides radio frequency current to the skin through the radio frequency circuit and the electrode units to make skin tissue generate heat. Furthermore, the main circuit board is electrically connected to the drive unit, the drive unit is coupled to the gear set, and the electrode units are connected to the gear set to be driven by the gear set to rotate, so as to respond to different usage scenarios and increase convenience of use.

What is claimed is:

1. A radio frequency beauty instrument, suitable for using on skin of a user, wherein the radio frequency beauty instrument comprises:
a housing;
a main circuit board disposed in the housing;
a gear set disposed in the housing;
a drive unit disposed in the housing and electrically connected to the main circuit board, wherein the drive unit is configured to drive the gear set; and
a radio frequency module comprising a radio frequency circuit disposed on the main circuit board and a plurality of electrode units exposed outside the housing and electrically connected to the radio frequency circuit, wherein the electrode units are connected to the gear set to be driven by the gear set to rotate, and the radio frequency module provides a radio frequency current to the skin through the radio frequency circuit and the electrode units.

2. The radio frequency beauty instrument as claimed in claim 1, wherein the electrode units respectively comprise a plurality of electrode disk bodies and a plurality of electrode protrusions located on the electrode disk bodies, and in response to the electrode units being driven by the gear set to rotate, a distance between the electrode protrusions changes.

3. The radio frequency beauty instrument as claimed in claim 2, wherein two of the electrode disk bodies have different diameters.

4. The radio frequency beauty instrument as claimed in claim 2, wherein two of the electrode disk bodies have same diameter.

5. The radio frequency beauty instrument as claimed in claim 1, wherein the gear set comprises a first gear and a plurality of second gears meshing with the first gear, the first gear is a driving gear, the second gears are driven gears, the drive unit drives the first gear, and the second gears are respectively driven to rotate the electrode units.

6. The radio frequency beauty instrument as claimed in claim 5, wherein the drive unit and the first gear are coaxial, and each of the second gears is coaxial with a corresponding one of the electrode units.

7. The radio frequency beauty instrument as claimed in claim 5, wherein the second gears surround the first gear, and a rotation direction of the first gear is opposite to a rotation direction of the second gears.

8. The radio frequency beauty instrument as claimed in claim 1, further comprising:
a sub circuit board electrically connected to the main circuit board, and disposed between the gear set and the electrode units.

9. The radio frequency beauty instrument as claimed in claim 8, wherein each of the electrode units comprises at least one spring pin connector, and each of the electrode units is electrically connected to the sub circuit board by abutting the at least one spring pin connector against the sub circuit.

10. The radio frequency beauty instrument as claimed in claim 9, wherein the sub circuit board comprises a plurality of ring pads, the ring pads respectively comprise at least one ring circuit, the ring circuit is a trajectory for each of the electrode units when rotating, and in response to the electrode units being driven, the spring pin connector of each of the electrode units is rotated along a corresponding one of the ring circuit.
